SEQUENCE LISTING (9 sequences)

SEQ ID NO: 1 - DNA, 118 bp, Artificial Sequence
  Synthetic aptamer
cgtgatgatgttgagttggccgagattgaagctggtgaccattggccttactgggatgctcttgtgattactactactaatcgttgtgaacgtaaatgccagtaatgccaaccaatct

SEQ ID NO: 2 - DNA, 118 bp, Artificial Sequence
  Synthetic aptamer
cgtgatgatgttgagttggtgttaatccgtgtatgccatgagaccggtaagtcctattttcgctcctggaatggtacaagctcatgtcaagatatactccagtaatgccaaccaatct

SEQ ID NO: 3 - DNA, 118 bp, Artificial Sequence
  Synthetic aptamer
cgtgatgatgttgagttggggcagatccgtgcacatgatcttagccgtacgtgttctgcggtatagtctattcatactaccacttcgcaatgtatatcgcagtaatgccaaccaatct

SEQ ID NO: 4 - DNA, 118 bp, Artificial Sequence
  Synthetic aptamer
cgtgatgatgttgagttggtcgagtacggatcacttggagattggccatcgagctattctgtaatgtgtaatcctactacaccttcgcatcgttaatgccagtaatgccaaccaatct

SEQ ID NO: 5 - DNA, 123 bp, Artificial Sequence
  Synthetic aptamer
cgtgatgatgttgagttggagttgcgttgctgaatgcatgggctgtactaggttggtgctactctgtaatgtctactattactattccatcgcaacgtatactgcagtaatgccaaccaatct

SEQ ID NO: 6 - DNA, 123 bp, Artificial Sequence
  Synthetic aptamer
cgtgatgatgttgagttggagttggggtgatgggtgcatgtgatgaaaggggttcgtgctatgctgttttgtctaataatactagtccttgccaaggtttattccagtaatgccaaccaatct

SEQ ID NO: 7 - DNA, 117 bp, Artificial Sequence
  Synthetic aptamer
  misc_feature (19)..(98): n is a, c, g, or t
cgtgatgatgttgagttgnnnnnnnnnnnnnnnnnnnnnnnnnnnnnnnnnnnnnnnnnnnnnnnnnnnnnnnnnnnnnnnnnnnnnnnnnnnnnnnncagtaatgccaaccaatct

SEQ ID NO: 8 - DNA, 18 bp, Artificial Sequence
  Synthetic primer
cgtgatgatgttgagttg

SEQ ID NO: 9 - DNA, 19 bp, Artificial Sequence
  Synthetic primer
agattggttggcattactg

(19) United States
(12) Patent Application Publication
(10) Pub. No.: US 2018/0292404 A1
(43) Pub. Date: Oct. 11, 2018

(54) ESCHERICHIA COLI O157:H7 APTAMER AND APPLICATIONS THEREOF

(71) Applicants: Mana Oloomi, Tehran (IR); Masoum Amraee, Tehran (IR)

(72) Inventors: Mana Oloomi, Tehran (IR); Masoum Amraee, Tehran (IR)

(21) Appl. No.: 15/927,680

(22) Filed: Mar. 21, 2018

Related U.S. Application Data
(60) Provisional application No. 62/485,905, filed on Apr. 15, 2017.

(51) Int. Cl. G01N 33/569; C12N 15/115

(52) U.S. Cl. CPC G01N 33/56911; C12N 2310/16; C12N 15/115

(57) ABSTRACT

A method for detecting Escherichia coli (E. coli) O157:H7 in a sample including synthesizing an E. coli O157:H7 aptamer, adding the E. coli O157:H7 aptamer to the sample allowing for formation of a complex by contact of the E. coli O157:H7 aptamer with any E. coli O157:H7 in the sample, and determining the formation of the complex by detecting the presence of the complex in the sample. The E. coli O157:H7 aptamer includes a nucleic acid sequence as set forth in one of SEQ ID No. 1, SEQ ID No. 2, SEQ ID No. 3, SEQ ID No. 4, SEQ ID No. 5, and SEQ ID No. 6.

### CROSS-REFERENCE TO RELATED APPLICATION

This application claims the benefit of priority from pending U.S. Provisional Patent Application Ser. No. 62/485,905, filed on Apr. 15, 2017, and entitled “DNA APTAMER FOR DETECTION OF ESCHERICHIA COLI O157,” which is incorporated herein by reference in its entirety.

### SPONSORSHIP STATEMENT

This application has been sponsored by Iran Patent Center, which does not have any rights in this application.

### TECHNICAL FIELD

The present disclosure generally relates to a method for detecting bacteria, particularly to a method for specifically detecting Escherichia coli O157:H7 using a DNA aptamer.

### BACKGROUND

Escherichia coli (E. coli) O157:H7 is a major foodborne pathogen that causes diseases such as hemorrhagic diarrhea, uremic hemorrhagic colitis, and kidney failure. These diseases have a significant impact on child mortality rates in developing countries. Therefore, rapid and accurate detection of E. coli O157:H7 contamination in samples is essential for aiding in avoiding toxic effects of E. coli O157:H7 and for treating infectious diseases of E. coli O157:H7.

Conventional methods for detecting E. coli O157:H7 in a sample entail utilizing classical cultures and serology. Recognition of E. coli O157:H7 using these methods is time-consuming and has a low specificity. Accordingly, there is a need for simple and cost-effective methods for detecting E. coli O157: H7 contamination in a cheaper, faster, and more effective manner.

### SUMMARY

This summary is intended to provide an overview of the subject matter of the exemplary embodiments of the present disclosure, and is not intended to identify essential elements or key elements of the subject matter, nor is it intended to be used to determine the scope of the claimed implementations. The proper scope of the exemplary embodiments of the present disclosure may be ascertained from the claims set forth below in view of the detailed description below and the drawings.

In one general aspect, the present disclosure describes an exemplary method for detecting Escherichia coli (E. coli) O157:H7 in a sample. The exemplary method may include synthesizing an E. coli O157:H7 aptamer, adding the E. coli O157:H7 aptamer to the sample allowing for formation of a complex by contact of the E. coli O157:H7 aptamer with any E. coli O157:H7 in the sample, and determining the formation of the complex by detecting the presence of the complex in the sample. In some exemplary embodiments, the aptamer may include a nucleic acid sequence as set forth in one of SEQ ID No. 1, SEQ ID No. 2, SEQ ID No. 3, SEQ ID No. 4, SEQ ID No. 5, and SEQ ID No. 6. In an exemplary implementation, the aptamer may include the nucleic acid sequence as set forth in SEQ ID No. 6.

The above general aspect may include one or more of the following features. In some exemplary implementations, synthesizing the E. coli O157:H7 aptamer may include identifying the E. coli O157:H7 aptamer, and amplifying the E. coli O157:H7 aptamer. In some exemplary implementations, identifying the E. coli O157:H7 aptamer may include identifying the E. coli O157:H7 aptamer using the whole-cell systematic evolution of ligands by exponential enrichment (cell-SELEX) method. In an exemplary implementation, amplifying the E. coli O157:H7 aptamer may include amplifying the E. coli O157:H7 aptamer using polymerase chain reaction (PCR).

According to some exemplary embodiments, the E. coli O157:H7 aptamer may have between about 118 and about 123 nucleotides. In some exemplary implementations, adding the E. coli O157:H7 aptamer to the sample allowing for formation of the complex by contact of the E. coli O157:H7 aptamer with any E. coli O157:H7 in the sample may include adding an aptamer solution with a concentration more than about 300 pM to the sample.

According to some exemplary implementations, determining the formation of the complex by detecting the presence of the complex in the sample may include detecting the presence of the complex using one of polymerase chain reaction (PCR), Real-Time PCR, an affinity tag, fluorescence, chemiluminescence, nuclear magnetic resonance, enzyme-linked immunosorbent assay (ELISA), and combinations thereof. In some exemplary implementations, determining the formation of the complex by detecting the presence of the complex in the sample may include detecting the quantity of the complex in the sample using an aptasensor.

According to some exemplary embodiments, the sample may include one of a physiological sample and a cultural sample. In some exemplary embodiments, the physiological sample may include one of saliva, urine, and blood. The physiological sample comprises one of a human physiological sample and an animal physiological sample. In some exemplary embodiments, the cultural sample may include one of a water sample or a food sample.

In another general aspect, the present disclosure describes an exemplary E. coli O157:H7 aptamer for binding to Escherichia coli (E. coli) O157:H7. The aptamer may include a nucleic acid sequence as set forth in one of SEQ ID No. 1, SEQ ID No. 2, SEQ ID No. 3, SEQ ID No. 4, SEQ ID No. 5, and SEQ ID No. 6.

The above general aspect may include one or more of the following features. In some exemplary embodiments, the aptamer may have a dissociation constant (K_{d}) between about 39.8 pM and about 175.4 pM. In some exemplary embodiments, the aptamer may have between about 118 and about 123 nucleotides. In some exemplary embodiments, the aptamer may bind to one of a cell wall and a cell membrane of the E. coli O157:H7. In some exemplary embodiments, the aptamer may be linked to one of a fluorescent tag, an affinity tag, a magnetic tag, a therapeutic agent, and combinations thereof.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a method for detecting Escherichia coli O157:H7 in a sample, consistent with one or more exemplary embodiments of the present disclosure.

FIG. 1B illustrates a method for synthesizing an Escherichia coli O157:H7 aptamer, consistent with one or more exemplary embodiments of the present disclosure.

FIG. 1C illustrates a method for identifying an Escherichia coli O157:H7 aptamer, consistent with one or more exemplary embodiments of the present disclosure.

FIG. 2 illustrates a schematic view of a secondary structure of an Escherichia coli O157:H7 aptamer, consistent with one or more exemplary embodiments of the present disclosure.

FIG. 3 illustrates an exemplary image of an agarose gel after electrophoresis of an amplified library of aptamers, consistent with one or more exemplary embodiments of the present disclosure.

FIG. 4 illustrates binding affinity of six candidates of E. coli O157:H7 aptamers, consistent with one or more exemplary embodiments of the present disclosure.

FIG. 5A illustrates percentage of fluorescence intensity of E. coli O157:H7 cells after incubation with different concentrations of E. coli O157:H7 aptamer, consistent with one or more exemplary embodiments of the present disclosure.

FIG. 5B illustrates the correlation between concentrations of E. coli O157:H7 aptamer and the percentage of fluorescent E. coli O157:H7 cells, consistent with one or more exemplary embodiments of the present disclosure.

FIG. 6 illustrates binding affinity of candidate E. coli O157:H7 aptamers after nine rounds of cell-SELEX and one round of counter-SELEX, and selected aptamer pool in the third rounds of cell-SELEX, consistent with one or more exemplary embodiments of the present disclosure.

FIG. 7A illustrates binding affinity of the E. coli O157:H7 aptamer with a nucleotide sequence as set forth in SEQ ID No. 6 (AM-6) to E. coli 042 cells, consistent with one or more exemplary embodiments of the present disclosure.

FIG. 7B illustrates binding affinity of the E. coli O157:H7 aptamer with a nucleotide sequence as set forth in SEQ ID No. 6 (AM-6) to E. coli K12 cells, consistent with one or more exemplary embodiments of the present disclosure.

FIG. 7C illustrates binding affinity of the E. coli O157:H7 aptamer with a nucleotide sequence as set forth in SEQ ID No. 6 (AM-6) to E. coli TOP10 cells, consistent with one or more exemplary embodiments of the present disclosure.

FIG. 7D illustrates binding affinity of the E. coli O157:H7 aptamer with a nucleotide sequence as set forth in SEQ ID No. 6 (AM-6) to E. coli DH5α cells, consistent with one or more exemplary embodiments of the present disclosure.

FIG. 7E illustrates binding affinity of the E. coli O157:H7 aptamer with a nucleotide sequence as set forth in SEQ ID No. 6 (AM-6) to Salmonella typhi cells, consistent with one or more exemplary embodiments of the present disclosure.

FIG. 7F illustrates binding affinity of the E. coli O157:H7 aptamer with a nucleotide sequence as set forth in SEQ ID No. 6 (AM-6) to Shigella flexneri cells, consistent with one or more exemplary embodiments of the present disclosure.

### DETAILED DESCRIPTION

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

Despite the development of different approaches, identification of specific bacteria in complex samples such as food remains a difficult challenge. Therefore, various immunological and molecular approaches have been developed which have aided in increasing sensitivity and specificity of the detection. Single-stranded nucleic acid aptamers represent a new generation of molecular approaches that have been developed for sensitive, specific, and rapid detection of target molecules. The ability of the aptamers to fold into unique and stable secondary structures helps them bind specifically to their specific targets such as bacteria.

In aptamer-based methods, after multiple rounds of selection and enrichment, secondary structural features of aptamers may be exploited for capturing specific target cell. Aptamers have several characteristics that make them a more attractive option for utilization than antibodies for developing biodiagnostic assays. These characteristics include a smaller size, ease of synthesis and renaturation, lack of immunogenicity, lack of batch to batch variation, lower cost of production, higher stability, and higher target specificity.

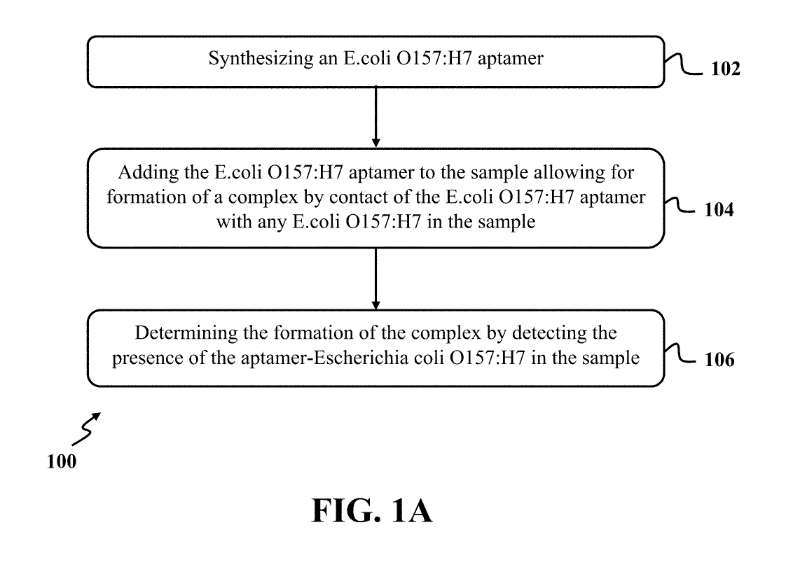

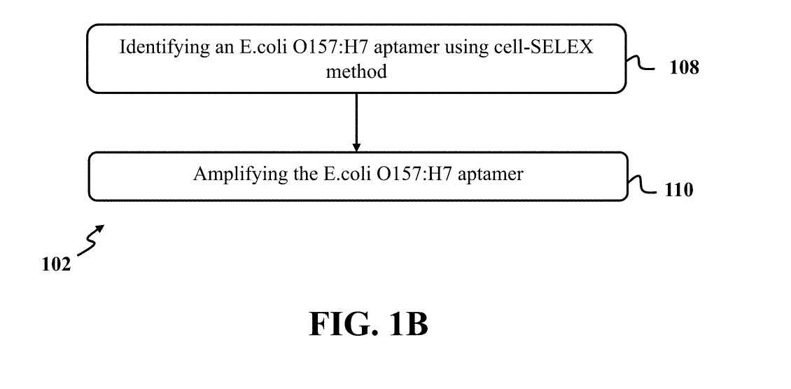

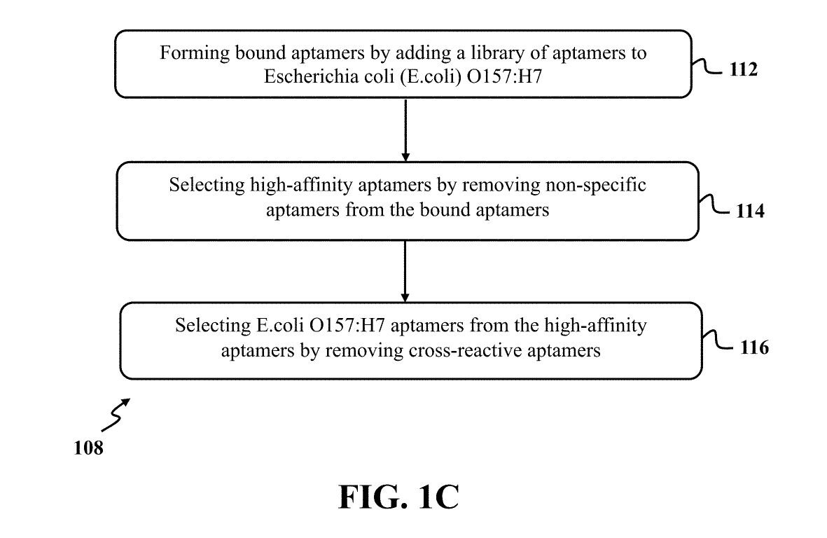

Disclosed herein is a simple and rapid method for detecting Escherichia coli (E. coli) O157:H7 using an aptamer which may specifically bind to the surface of E. coli O157:H7 cells in a sample. FIG. 1A is method 100 for detecting E. coli O157:H7 in the sample, consistent with one or more exemplary embodiments of the present disclosure. Method 100 may include synthesizing an E. coli O157:H7 aptamer (step 102), adding the E. coli O157:H7 aptamer to the sample allowing for formation of a complex by contact of the E. coli O157:H7 aptamer with any E. coli O157:H7 in the sample (step 104), and determining the formation of the complex by detecting the presence of the complex in the sample (step 106).

FIG. 1B shows an exemplary implementation of step 102 for synthesizing the E. coli O157:H7 aptamer, consistent with one or more exemplary embodiments of the present disclosure. Synthesizing the E. coli O157:H7 aptamer may include identifying the E. coli O157:H7 aptamer (step 108) and amplifying the E. coli O157:H7 aptamer (step 110).

Step 108 may include identifying the E. coli O157:H7 aptamer. The E. coli O157:H7 aptamer may be identified after several rounds of the whole-cell systematic evolution of ligands by exponential enrichment (cell-SELEX) method. The cell-SELEX method may be used for aptamer selection as an alternative to the SELEX method which may be applied to crude or purified extracellular surface of targets. The advantage of the cell-SELEX method over the conventional SELEX method is that in the cell-SELEX method there is no need for detailed knowledge about the target to start the selection. Selection procedure may be applied to the target cells in their original conformation and physiological condition.

It should be noted that in the cell-SELEX method, an iterative process of in-vitro selection of specific and high-affinity aptamers may be done using a large random-sequence library of aptamers. After conducting the cell-SELEX method, the identified E. coli O157:H7 aptamers may show a high specificity and a high affinity for E. coli O157:H7 bacteria.

FIG. 1C shows an exemplary implementation of step 108 for identifying the E. coli O157:H7 aptamer using the cell-SELEX method, consistent with one or more exemplary embodiments of the present disclosure. Identifying the E. coli O157:H7 aptamer may include forming bound aptamers by adding a library of aptamers to E. coli O157:H7 (step 112), selecting high-affinity aptamers by removing non-specific aptamers from the bound aptamers (step 114), and selecting E. coli O157:H7 aptamers from the high-affinity aptamers by removing cross-reactive aptamers (step 116).

It should be noted that the cell-SELEX method may include several iterations of aptamer enrichment by incubating the library of aptamers with the E. coli O157:H7 cells followed by separation of bound aptamers from unbound aptamers. Candidate aptamers may be enriched by PCR amplification. The amplified candidate aptamers may be subjected to further rounds of cell-SELEX.

Step 112 may include forming bound aptamers by adding the library of aptamers to the E. coli O157:H7. In one or more exemplary implementations, the library of aptamers with random sequences may be chemically synthesized. It should be noted that the library of aptamers may be expected to have between about 10¹⁴ and about 10¹⁶ unique aptamer sequences theoretically. In one or more exemplary embodiments, the synthesized library of aptamers may be purified using polyacrylamide gel electrophoresis (PAGE) method.

In some exemplary embodiments, each aptamer in the library of aptamers may be a single-stranded oligonucleotide which may include a central random region with between about 81 and about 86 nucleotides flanked by two overhangs with different constant sequences at 5′ and 3′ ends. In one or more exemplary embodiments, the overhangs may act as binding regions of primers for subsequent amplification. In one or more exemplary embodiments, the random aptamers have a nucleotide sequence as set forth in SEQ ID No. 7.

In some exemplary embodiments, the overhang at the 5′ end may be a binding region for a forward primer. The forward primer may have a nucleotide sequence as set forth in SEQ ID No. 8. In some exemplary embodiments, the overhang at the 3′ end may be a binding region for a reverse primer. The reverse primer may have a nucleotide sequence as set forth in SEQ ID No. 9. In some exemplary embodiments, the forward and reverse primers may be labeled with one of a fluorescent tag, an affinity tag, and a magnetic tag. In one or more exemplary embodiments, the fluorescent tag may include one of fluorescein isothiocyanate (FITC), and fluorescein (FAM). In one or more exemplary embodiments, the affinity tag may include streptavidin/biotin tag.

In some exemplary embodiments, the aptamers of the library may be amplified using a labeled forward primer and a labeled reverse primer in a polymerase chain reaction (PCR). In some exemplary embodiments, the PCR for labeling and amplification of the aptamers may be conducted using labeled primers. In one or more exemplary embodiments, after conducting the PCR, the amplified aptamers may be purified using a PCR purification kit. The quality and size of the amplified aptamers as the PCR products may be confirmed using agarose gel electrophoresis.

In some exemplary implementations, after amplification the aptamers of the library, single-stranded aptamers may be obtained through an asymmetric PCR with a modified concentration ratio of the forward and the reverse primers. In some exemplary embodiments, after conducting the asymmetric PCR, the labeled single-stranded aptamers of the forward or reverse strand may be separated and the single-stranded aptamers of another strand may be discarded. In one or more exemplary embodiments, the FITC-labeled single-stranded aptamers may be separated and the single-stranded aptamers of another strand may be discarded.

In some exemplary implementations, in the first round of cell-SELEX, the labeled single-stranded aptamers may be added to E. coli O157:H7. It should be noted that by incubating the labeled single-stranded aptamers with the E. coli O157:H7 cells, some labeled single-stranded aptamers may have an affinity for E. coli O157:H7 and may be able to bind to the E. coli O157:H7 cells and form bound aptamers due to their secondary structural feature.

In one or more exemplary embodiments, prior to adding the labeled single-stranded aptamers to the E. coli O157:H7, the labeled single-stranded aptamers may be dissolved in a binding buffer including sodium chloride (NaCl), magnesium chloride (MgCl₂), Tris-hydrochloride (Tris-HCl), and potassium chloride (KCl). In some exemplary embodiments, the labeled single-stranded aptamers may be incubated with the E. coli O157:H7 cells at a temperature of about 25 C for a time period of about 60 minutes with mild shaking to form bound aptamers. In one or more exemplary embodiments, the E. coli O157:H7 cells may be cultured in a broth medium until the middle growth phase. The cultured E. coli O157:H7 bacteria may be centrifuged and the supernatant may be removed.

Step 114 may include selecting high-affinity aptamers by removing non-specific aptamers from the bound aptamers. In some exemplary embodiments, bound and unbound aptamers may be partitioned by washing and centrifugation of the E. coli O157:H7 cells. In some exemplary embodiments, the unbound aptamers may be removed from the bound aptamers by adding a washing buffer, centrifuging, removing the supernatant, and suspending the pellet in distilled water (DW). In one or more exemplary embodiments, the washing buffer may include phosphate-buffered-saline (PBS), glucose, and magnesium chloride (MgCl₂) with a concentration of about 1M.

In one or more exemplary embodiments, after separating bound aptamers from the library of aptamers, the bound aptamers may be eluted and separated from the E. coli O157:H7 cells in a heating and cooling process. In the heating and cooling process, the bound aptamers may be heated to a temperature of about 94° C. for a time period of about 10 minutes and then placed on ice for a time period about 10 minutes. In some exemplary embodiments, after eluting the bound aptamers, the eluate may be centrifuged with a speed of about 13000 rounds per minute (RPM) for a time period of about 10 minutes and the supernatant may be isolated and designated as candidate aptamers.

It should be noted that, after performing the first round of cell-SELEX and obtaining the candidate aptamers, several further rounds of cell-SELEX may be performed on the candidate aptamers to enrich them and find high-affinity aptamers. In some exemplary embodiments, in order to perform other rounds of cell-SELEX, the eluted bound aptamers may be divided into several samples, and candidate aptamers of each sample may be used for amplification by PCR, and the amplified candidate aptamers may be used for next round of cell-SELEX selection.

In one or more exemplary embodiments, about nine rounds of cell-SELEX may be directed against E. coli O157:H7. It should be noted that after each round of cell-SELEX, the candidate aptamers may be enriched and the affinity of the candidate aptamers for E. coli O157:H7 may be increased. In some exemplary embodiments, nine selection rounds of cell-SELEX may be performed until high-affinity aptamers may be obtained. The candidate aptamers of the ninth round of cell-SELEX may be high-affinity aptamers for detection and capturing of E. coli O157:H7 cells.

Step 116 may include selecting E. coli O157:H7 aptamers from the high-affinity aptamers by removing cross-reactive aptamers. It should be noted that some of the high-affinity aptamers may be cross-reactive and have a high affinity towards other bacterial cells. In some exemplary embodiments, E. coli O157:H7 aptamers may be selected by performing counter-SELEX selection against non-target bacterial cells and removing cross-reactive aptamers. In the counter-SELEX selection, the E. coli O157:H7 aptamers are without any affinity for other bacterial strains.

In some exemplary embodiments, the counter-SELEX selection may be directed against other strains of bacteria such as O42, K12, Top10, and DH5α E. coli cells, Shigella flexneri, and Salmonella typhi as negative selection counterparts. The counter-SELEX selection may increase the specificity of aptamer sequences.

It should be noted that manipulating the number and sequence of cell-SELEX and counter-SELEX iterations may lead to the identification of E. coli O157:H7 aptamers with a high degree of binding specificity. In some exemplary embodiments, in order to characterize the E. coli O157:H7 aptamers, the selected E. coli O157:H7 aptamers may be amplified, cloned, and sequenced.

In some exemplary embodiments, the E. coli O157:H7 aptamers may have a nucleotide sequence as set forth in one of SEQ ID No. 1, SEQ ID No. 2, SEQ ID No. 3, SEQ ID No. 4, SEQ ID No. 5, and SEQ ID No. 6. In one or more exemplary embodiments, the E. coli O157:H7 aptamers may have a nucleotide sequence as set forth in SEQ ID No. 6. In one or more exemplary embodiments, the E. coli O157:H7 aptamer may have a dissociation constant (K_{d}) between about 39.8 pM and about 175.4 pM.

It should be noted that the E. coli O157:H7 aptamers may specifically bind to surface of E. coli O157:H7 cells and selectively distinguishes the pathogenic strain of E. coli O157:H7 from other strains. In one or more exemplary embodiments, the aptamer may bind to one of a cell wall and a cell membrane of the E. coli O157:H7. In some exemplary embodiments, the E. coli O157:H7 aptamers may specifically detect the E. coli O157:H7 bacteria as a diagnostic agent and may be used for detection of E. coli O157:H7 foodborne diseases.

Referring back to FIG. 1B, step 110 may include amplifying the E. coli O157:H7 aptamer. In some exemplary embodiment, after identifying the E. coli O157:H7 aptamers, the E. coli O157:H7 aptamers may be amplified. In one or more exemplary embodiments, amplification of the E. coli O157:H7 aptamers may be done in a polymerase chain reaction (PCR) using the forward and reverse primers. In one or more exemplary embodiments, the forward primer may have a nucleotide sequence as set forth in SEQ ID No. 8. In some exemplary embodiments, the reverse primer may have a nucleotide sequence as set forth in SEQ ID No. 9.

Referring back to FIG. 1A, step 104 may include adding the E. coli O157:H7 aptamer to the sample allowing for formation of the complex by contact of the E. coli O157:H7 aptamer with any E. coli O157:H7 in the sample. The complex may include the E. coli O157:H7 aptamer which binds to the E. coli O157:H7 cells. In some exemplary embodiments, the complex of the E. coli O157:H7 aptamer and the E. coli O157:H7 cell may be formed after adding the E. coli O157:H7 aptamer to the sample responsive to binding of the E. coli O157:H7 aptamer to the E. coli O157:H7 cells.

In some exemplary embodiments, after synthesizing and amplifying the E. coli O157:H7 aptamer, the E. coli O157:H7 aptamer may be incubated with the sample at a temperature of about 25 C for a time period of about 60 minutes with mild shaking.

In some exemplary embodiments, the E. coli O157:H7 aptamer may specifically target the E. coli O157:H7 cells in the sample, if any, and may form the complex in the sample. The complex may include the E. coli O157:H7 aptamer which may specifically bind to surface of the E. coli O157:H7 cells. In one or more exemplary embodiments, adding the E. coli O157:H7 aptamer to the sample allowing for formation of the complex by contact of the E. coli O157:H7 aptamer with any E. coli O157:H7 in the sample may include adding an aptamer solution with a concentration more than about 300 pM to the sample. In some exemplary embodiments, the E. coli O157:H7 aptamer may be linked to one of a fluorescent tag, an affinity tag, a magnetic tag, a therapeutic agent, and combinations thereof.

In some exemplary embodiments, the sample may be one of the physiological sample and cultural sample. In one or more exemplary embodiments, the physiological sample may include one of saliva, urine, and blood. In one or more exemplary embodiments, the cultural sample may include one of a water sample, and a food sample. In one or more exemplary embodiments, the physiological sample my include one of a human physiological sample and an animal physiological sample.

Step 106 may include determining the formation of the complex by detecting the presence of the complex in the sample. The complex may be formed if there is any E. coli O157:H7 cells in the sample. In some exemplary embodiments, detecting the presence of the complex in the sample may include detecting the presence of the complex using one of polymerase chain reaction (PCR), Real-Time PCR, an affinity tag, fluorescence, chemiluminescence, nuclear magnetic resonance, enzyme-linked immunosorbent assay (ELISA), and combinations thereof.

In one or more exemplary embodiments, determining the formation of the complex by detecting the presence of the complex in the sample may include detecting the presence or quantity of the complex in the sample using an aptasensor. In some exemplary embodiments, the E. coli O157:H7 aptamers may be used for treating a disease caused by E. coli O157:H7 in a patient by administering an aptamer-based drug to the patient.

In some exemplary embodiments, the aptamer-based drug may include the E. coli O157:H7 aptamer linked to a therapeutic agent. The aptamer may have a nucleotide sequence as set forth in one of SEQ ID No. 1, SEQ ID No. 2, SEQ ID No. 3, SEQ ID No. 4, SEQ ID No. 5, and SEQ ID No. 6. In one or more exemplary embodiments, the disease caused by Escherichia coli O157:H7 may include one of hemorrhagic diarrhea, kidney failure, and combinations thereof. In some exemplary embodiments, the therapeutic agent may include one of the anti-Shiga toxin antibodies, antibiotics, and combinations thereof.

### EXAMPLES

### Example 1: Identifying E. Coli O157:H7 Aptamer Using the Cell-Selex Method

In this example, an E. coli O157:H7 aptamer was identified using the cell-SELEX method through the steps of forming bound aptamers by adding a library of aptamers to E. coli O157:H7, selecting high-affinity aptamers by removing non-specific aptamers from the bound aptamers, and selecting E. coli O157:H7 aptamers from the high-affinity aptamers by removing cross-reactive aptamers.

At first, the library of aptamers was added to E. coli O157:H7 cells to form bound aptamers. The library of aptamers with random sequences was chemically synthesized, and purified using the polyacrylamide gel electrophoresis (PAGE) method. Each aptamer in the library of aptamers had a single-stranded oligonucleotide as set forth in SEQ ID No. 7 with a central random region with between about 81 and about 86 nucleotides flanked by two overhangs with different constant sequences at 5′ and 3′ ends. The overhang at the 5′ end was a binding region for a forward primer with a nucleotide sequence as set forth in SEQ ID No. 8. Also, the overhang at the 3′ end was a binding region for the reverse primer with a nucleotide sequence as set forth in SEQ ID No. 9.

In order to obtain labeled single-stranded aptamers, the aptamers of the library were amplified using a labeled forward primer and a labeled reverse primer in a polymerase chain reaction (PCR). The forward primer was labeled with fluorescein isothiocyanate (FITC), and the reverse primer was labeled with biotin. The polymerase chain reaction (PCR) for labeling and amplification of the aptamers was conducted using a thermal program. TABLE. 1 shows the thermal program of the PCR for labeling and amplification of the aptamers which includes denaturation, annealing, extension, and final extension.

TABLE 1
Thermal protocol of the PCR for labeling
and amplification of the aptamers
Cycles 20 Cycles from step 2 to 4

| Step No. | | Time | Temperature |
|---|---|---|---|
| 1 | Initial denaturation | 5 min | 95° C. |
| 2 | Denaturation | 1 min | 95° C. |
| 3 | Annealing | 1 min | 55° C. |
| 4 | Extension | 1 min | 72° C. |
| 5 | Final extension | 10 min | 72° C. |

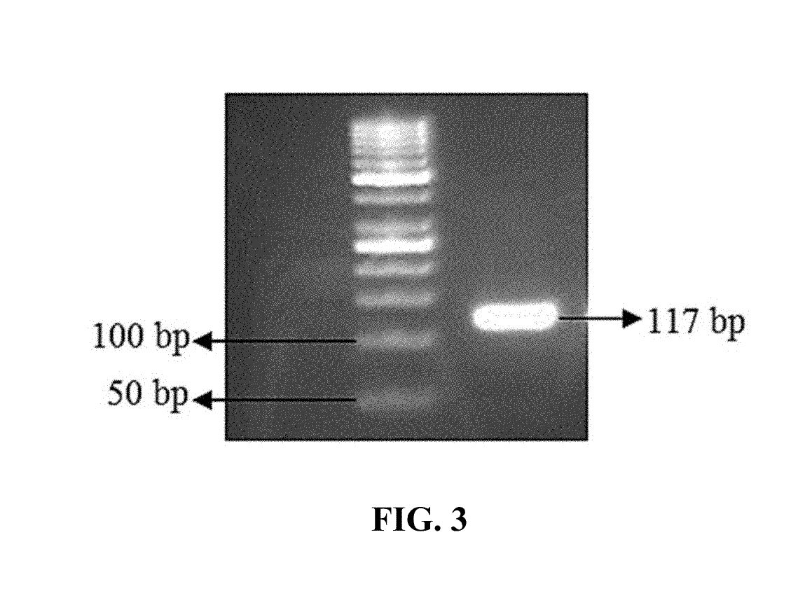

After conducting the PCR, the amplified labeled double-stranded aptamers were purified using a PCR purification kit. The quality and size of the PCR products were confirmed using agarose gel electrophoresis. FIG. 3 shows an exemplary image of an agarose gel after electrophoresis of the amplified library of aptamers, consistent with one or more exemplary embodiments of the present disclosure. Referring to FIG. 3, the PCR products show a band in the agarose gel with a molecular size of about 117 base pairs (bp) which confirms the amplification of the double-stranded aptamers with a nucleotide sequence as set forth in SEQ ID No. 7.

After amplification the aptamers of the library, the single-stranded aptamers were obtained using an asymmetric PCR. In the asymmetric PCR, the labeled forward and labeled reverse primers were used with a molar ratio of about 4:1 (forward primer:reverse primer). After conducting the asymmetric PCR, the FITC-labeled single-stranded aptamers were separated by discarding the biotin-labeled reverse strand. The E. coli O157:H7 bacteria was cultured in a broth medium until the middle growth phase with an optical density of about 0.6 at a wavelength of about 600 nm. Then, the cultured E. coli O157:H7 bacteria was centrifuged at a speed of about 13000 round per minute (RPM) for a time period about 10 minutes to remove the supernatant.

After that, the FITC-labeled single-stranded aptamers were added to E. coli O157:H7 to form bound aptamers. The FITC-labeled single-stranded aptamers with an amount of about 400 pmol was dissolved in 900 ml of a binding buffer to form the aptamer solution. The binding buffer includes 100 mM NaCl, 1 mM MgCl₂, 50 mM Tris-HCl, 5 mM KCl. After dissolving the FITC-labeled single-stranded aptamers, the aptamer solution was added to about 10⁷ E. coli O157:H7 cells and incubated at a temperature of about 25 C for a time period of about 60 minutes with mild shaking. During the incubation, some FITC-labeled single-stranded aptamers were able to bind to the E. coli O157:H7 bacteria and form bound aptamers.

In the next step, bound aptamers were separated from the library of aptamers by removing non-specific and unbound aptamers from the bound aptamers. The bound and unbound aptamers were partitioned by washing and centrifugation of the E. coli O157:H7 cells. The unbound aptamers were removed from the bound aptamers by adding a washing buffer, centrifuging at 13000 RPM for a time period about 10 minutes, removing the supernatant, and suspending the pellet in distilled water (DW). The washing buffer includes 1 L phosphate-buffered saline (PBS), 4.5 gram of glucose, and 5 ml of magnesium chloride (MgCl₂) with a concentration of about 1M.

After that, the bound aptamers were eluted and separated from the E. coli O157:H7 cells in a heating and cooling process. In the heating and cooling process, the bound aptamers were heated to a temperature of about 94° C. for a time period of about 10 minutes and then placed on ice for a time period about 10 minutes. After eluting the bound aptamers, the eluate was centrifuged with a speed of about 13000 RPM for a time period of about 10 minutes and the supernatant was isolated and designated as candidate aptamers.

After performing the first round of cell-SELEX and obtaining the candidate aptamers, enrichment of the candidate aptamers was done by performing nine rounds of cell-SELEX on the candidate aptamers. In order to perform further rounds of cell-SELEX, the candidate aptamers were divided into 10 samples. Each sample including candidate aptamers was used for amplification by PCR, and the amplified candidate aptamers were used for next rounds selection against E. coli O157:H7. After performing further rounds of cell-SELEX, high-affinity aptamers were selected as an ideal aptamer for detection and capturing of E. coli O157:H7.

In the next step, E. coli O157:H7 aptamers were selected from the high-affinity aptamers by removing cross-reactive aptamers. Some of the high-affinity aptamers were cross-reactive and have a high affinity towards other bacterial cells, such as O42, K12, Top10, and DH5α E. coli cells, Shigella flexneri, and Salmonella typhi as negative selection counterparts.

In this step, E. coli O157:H7 aptamers were selected by performing counter-SELEX selection against non-target bacterial cells and removing cross-reactive aptamers. The counter-SELEX selection was directed against other strains of bacteria such as O42, K12, Top10, and DH5α E. coli cells, Shigella flexneri, and Salmonella typhi to enrich the specificity of the E. coli O157:H7 aptamers. In the counter-SELEX selection, the E. coli O157:H7 aptamers are without any affinity for other bacterial strains.

After performing the counter-SELEX selection and selecting the E. coli O157:H7 aptamers, in order to characterize the E. coli O157:H7 aptamers, the selected E. coli O157:H7 aptamers were amplified in PCR, cloned, and then sequenced. The E. coli O157:H7 aptamers were cloned using the pTZ57R/T cloning kit. Transformed colonies with inserts of E. coli O157:H7 aptamer were selected using blue/white screening method on LB plates containing ampicillin and Xgal. Plasmid extraction was done and subsequently nucleotide sequencing was performed.

The E. coli O157:H7 aptamers had a nucleotide sequence as set forth in one of SEQ ID No. 1, SEQ ID No. 2, SEQ ID No. 3, SEQ ID No. 4, SEQ ID No. 5, and SEQ ID No. 6. The E. coli O157:H7 aptamer with a nucleotide sequence as set forth in SEQ ID No. 6 has a higher affinity for the E. coli O157:H7 cells, and has a definite stem-loop structure in the random region. Also, the E. coli O157:H7 aptamer had a dissociation constant (K_{d}) between about 39.8 pM and about 175.4 pM.

The E. coli O157:H7 aptamers specifically bind to the surface of E. coli O157:H7 cells and selectively distinguish the pathogenic strain of E. coli O157:H7 from other strains. The E. coli O157:H7 aptamers specifically detect the E. coli O157:H7 bacteria as a diagnostic agent for detecting E. coli O157:H7 foodborne diseases.

### Example 2: Secondary Structure of the E. Coli O157:H7 Aptamer

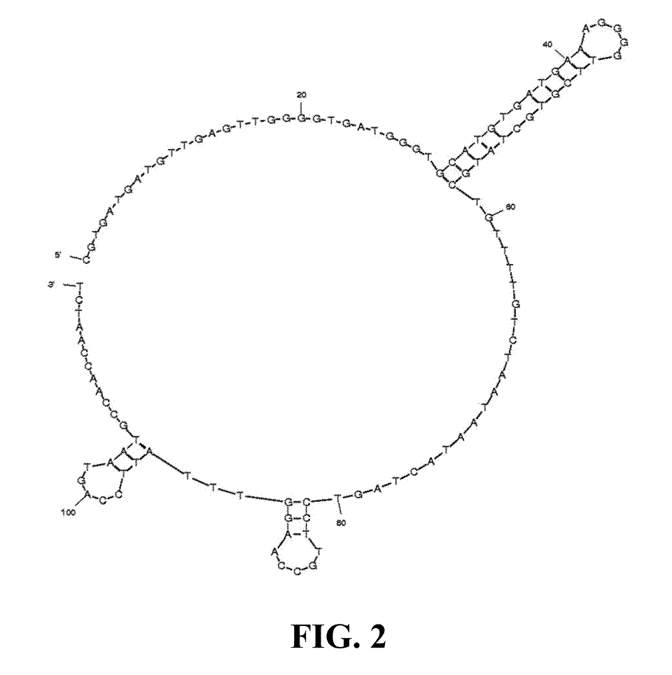

In this example, the secondary structure of the E. coli O157:H7 aptamer with a nucleotide sequence as set forth in SEQ ID No. 6 was predicted using the Mfold software. FIG. 2 shows a schematic view of a secondary structure of the E. coli O157:H7 aptamer with a nucleotide sequence as set forth in SEQ ID No. 6, consistent with one or more exemplary embodiments of the present disclosure. The secondary structure of the E. coli O157:H7 aptamer, that displayed a high affinity to E. coli O157:H7 cells, is predicted using the Mfold software which serves free energy minimization algorithms to predict folding structures.

Referring to FIG. 2, the predicted three dimensional folding of the E. coli O157:H7 aptamer with a nucleotide sequence as set forth in SEQ ID No. 6 includes three helices and an external loop. It has been suggested that defined loop-stem structure improves the stability of the aptamer. The E. coli O157:H7 aptamer with a nucleotide sequence as set forth in SEQ ID No. 6 has a definite stem-loop structure in the random region. The first helix consists of a hairpin loop with a closing pair between nucleotide 94e106 (N94-N106). The second and the third helices consist of an internal loop with an external closing pair between different nucleotides of N80-N92 and N29eN59, respectively.

Moreover, the minimum free energy of the E. coli O157:H7 aptamer with a nucleotide sequence as set forth in SEQ ID No. 6 was predicted as follows: ΔG°=−4.66 kcal/mol at a temperature of about 37° C. Although the Mfold software is a useful tool for the finding of binding motif or improvement of binding performance, this tool has several limitations to guarantee exact natural folding structure of aptamers. Therefore, in order to cope with this limitation, some lab experiments such as point mutation or truncation of aptamer sequences are required to verify the relevance of prediction.

### Example 3: Binding Affinity of the E. Coli O157:H7 Aptamer

In this example, binding affinity and dissociation constant (K_{d}) of the E. coli O157:H7 aptamers was evaluated. Preliminary binding affinity assays were performed by incubating 400 pmol FITC-labeled E. coli O157:H7 aptamers on 10⁷ intact cells of E. coli O157:H7 bacteria and were analyzed using flow cytometry using a FACS flow cytometer. Data from the FACS flow cytometer was analyzed using BD Cell-Quest™ Pro software.

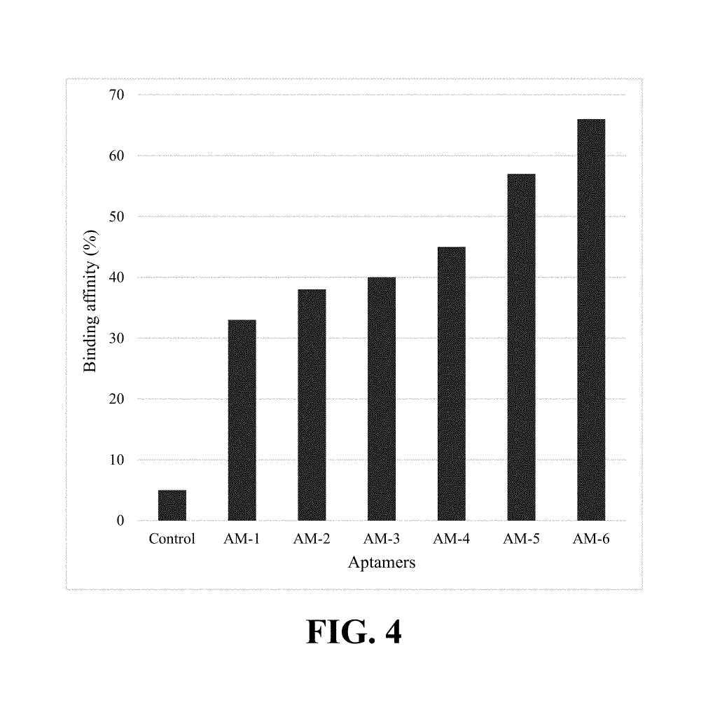

FIG. 4 shows binding affinity of six candidates of E. coli O157:H7 aptamers, consistent with one or more exemplary embodiments of the present disclosure. The six E. coli O157:H7 aptamers have a nucleotide sequence as set forth in SEQ ID No. 1 (AM-1), SEQ ID No. 2 (AM-2), SEQ ID No. 3 (AM-3), SEQ ID No. 4 (AM-4), SEQ ID No. 5 (AM-5), and SEQ ID No. 6 (AM-6).

Referring to FIG. 4, an approximate four to eight-fold increases in binding affinity was observed for the candidate E. coli O157:H7 aptamers following nine rounds of cell-SELEX and one round of counter-SELEX in comparison with the control group. AM-5 and AM-6 have the highest binding affinity in comparison to control and other aptamers, and the AM-1 shows the lowest binding affinity.

Moreover, dissociation constant (K_{d}) of the E. coli O157:H7 aptamer with a nucleotide sequence as set forth in SEQ ID No. 6 was calculated. The dissociation constant of the E. coli O157:H7 aptamer means the affinity between the E. coli O157:H7 aptamer and the E. coli O157:H7 bacteria. In comparison with the previous E. coli aptamers which have a dissociation constant in nanomolar (nM) scale, the exemplary E. coli O157:H7 aptamer of the present disclosure has a dissociation constant in picomolar (pM) scale. Therefore, the exemplary E. coli O157:H7 aptamer of the present disclosure shows higher binding affinity to E. coli O157:H7 bacteria.

One of the major limitations in the calculation of K_{d} is using a non-interacting model of binding sites with the non-linear square method which is based on the overall change in the number of fluorescently labeled cells. Consequently, it is impossible to predict the number of individual aptamer recognition sites residing on the surface of an individual cell. The specific binding of aptamers on different E. coli strains is related to specific components of the outer cellular membrane such as lipopolysaccharide (LPS), outer membrane proteins, and flagella.

The targets on the surface of the E. coli O157:H7 cells for each aptamer may be identified by aptamer-facilitated biomarker discovery (AptaBiD) method during the post-SELEX process. However, in the cell-SELEX method, a specific aptamer may bind to different targets in the surface of the bacterial cell with various affinities. Therefore, the result would be an aptamer with more specificity for precise bacterial detection.

Equilibrium dissociation constant (K_{d}) of the E. coli O157:H7 aptamer with a nucleotide sequence as set forth in SEQ ID No. 6 was determined using different concentrations of E. coli O157:H7 aptamer in a binding buffer and incubated with 10⁷ E. coli O157:H7 cells at a temperature of about 25° C. for a time period about 45 minutes with mild shaking. The concentrations of the E. coli O157:H7 aptamer with a nucleotide sequence as set forth in SEQ ID No. 6 was 50 pM, 150 pM, and 300 pM.

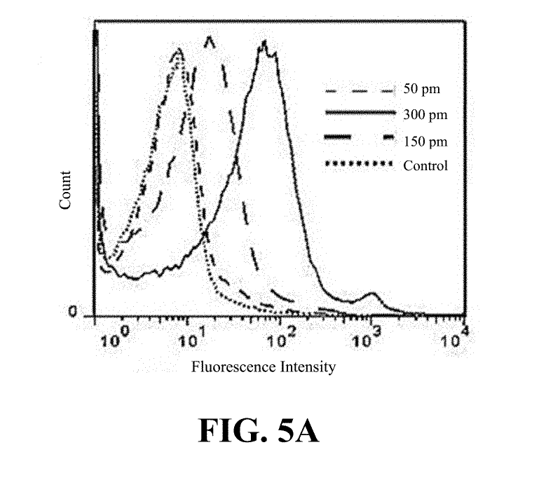

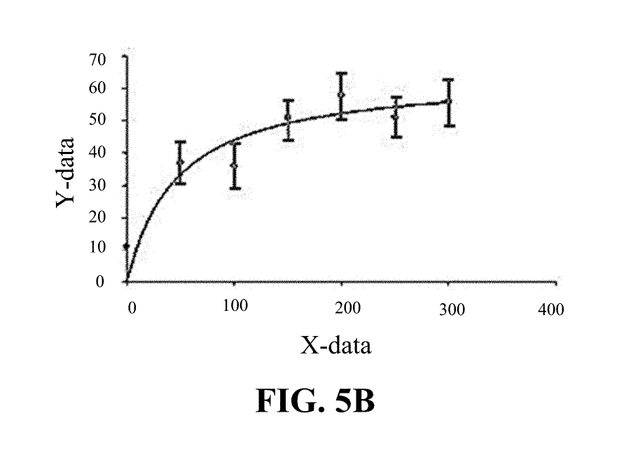

FIG. 5A shows percentage of fluorescence intensity of E. coli O157:H7 cells after incubation with different concentrations of E. coli O157:H7 aptamer with a nucleotide sequence as set forth in SEQ ID No. 6, consistent with one or more exemplary embodiments of the present disclosure. FIG. 5B shows correlation between concentrations of E. coli O157:H7 aptamer with a nucleotide sequence as set forth in SEQ ID No. 6 and the percentage of fluorescent E. coli O157:H7 cells, consistent with one or more exemplary embodiments of the present disclosure. X-Data is aptamer concentrations (pM) and Y-Data is relative aptamer binding based on fluorescence intensity.

Referring to FIGS. 5A and 5B, a total number of fluorescent cells increased by raising the concentration of FITC-labeled aptamer and reached a plateau based on the average fluorescence intensity per cell. The equilibrium dissociation constant (K_{d}) of the E. coli O157:H7 aptamer was determined by fitting the data of FIG. 5B a model. The equilibrium dissociation constant (K_{d}) was calculated in SigmaPlot by the following model: Y=BₘₐₓX/(K_{d}+X). Y is the average total percentage of bacterial fluorescent cells due to the binding of the FITC-labeled E. coli O157:H7 aptamer. X is considered as aptamer concentration and Bₘₐₓ is the maximum binding capacity of the E. coli O157:H7 aptamer. The calculated K_{d} of the E. coli O157:H7 aptamer was between about 39.8 pM and about 174.8 pM.

### Example 4: Specificity Analysis of the E. Coli O157:H7 Aptamer

In this example, specificity of the E. coli O157:H7 aptamer with a nucleotide sequence as set forth in SEQ ID No. 5 (AM-5) and in SEQ ID No. 6 (AM-6) were evaluated based on the results of binding affinity of the E. coli O157:H7 aptamers to E. coli O157:H7 during the preliminary screening in EXAMPLE 3.

At First, the E. coli O157:H7 aptamer was amplified in the PCR reactions using the FITC-labeled forward primer and biotin-labeled reverse primer. The FITC-labeled aptamer sequences were separated using alkaline denaturation. After that, about 400 pmol of FITC-labeled E. coli O157:H7 aptamers AM-5 and AM-6 were incubated with 10⁷ intact cells of E. coli O157:H7 bacteria. Finally, the fluorescence intensity of bacterial cells was analyzed using flow cytometry using a FACS flow cytometer. The mean fluorescence intensity (MFI) and percentage of fluorescent bacterial cells due to the E. coli O157:H7 aptamer binding were determined.

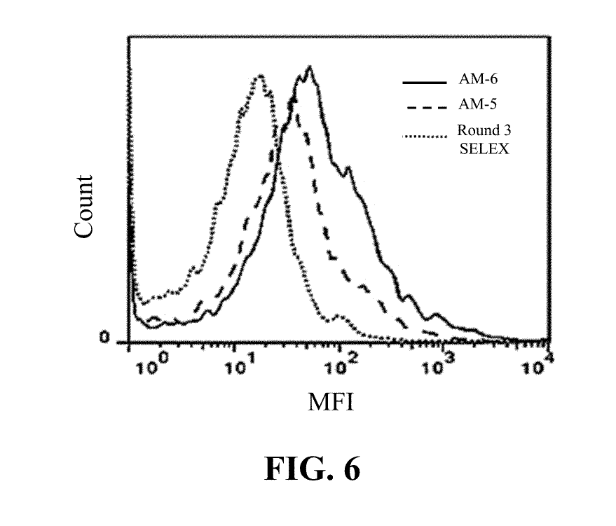

FIG. 6 shows binding affinity of candidate E. coli O157:H7 aptamers after nine rounds of cell-SELEX and one round of counter-SELEX, and selected aptamer pool in the third rounds of cell-SELEX, consistent with one or more exemplary embodiments of the present disclosure. Referring to FIG. 6, aptamers Am-5 and Am-6 were shown 57.41% and 66.26% of fluorescence intensity respectively, while the remaining aptamer pool from third rounds of cell-SELEX was shown only 5.13% of fluorescence intensity in the flow cytometry analysis.

### Example 5: Cross-Reactivity Assay of the E. Coli O157:H7 Aptamer

In this example, cross-reactivity of the E. coli O157:H7 aptamer was evaluated by determining the binding affinity of the E. coli O157:H7 aptamer to other bacteria such as O42, K12, Top 10 and DH5a, Shigella flexneri, and Salmonella typhi. The E. coli O157:H7 aptamer with a SEQ ID No. 6 (AM-6) was selected for this cross-reactivity assay, because the AM-6 aptamer shows a higher binding affinity and specificity, and has a definite stem-loop structure which makes this aptamer more stable. At first, the FTIC-labeled E. coli O157:H7 aptamer was incubated with 10⁷ mixed cells of O42, K12, Top10 and DH5α E. coli strains and the fluorescence intensity were analyzed using flow cytometry.

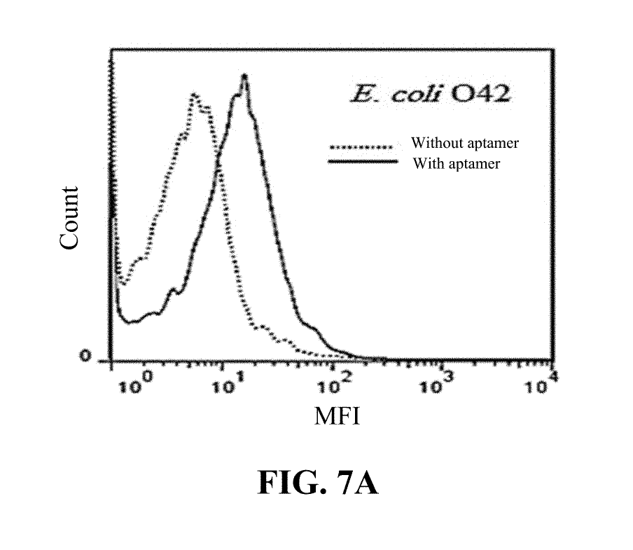

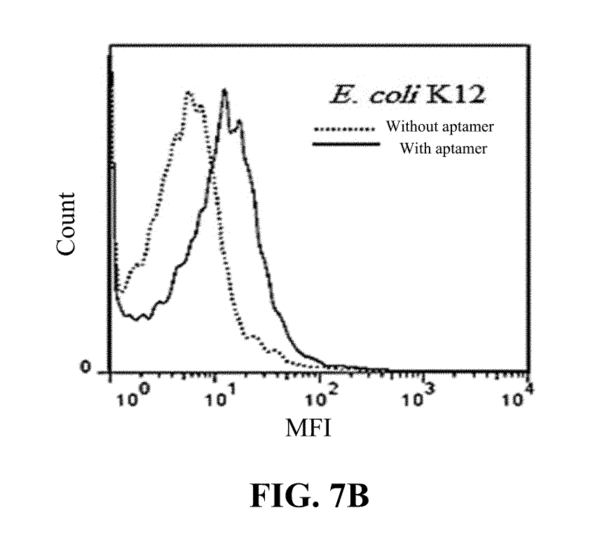

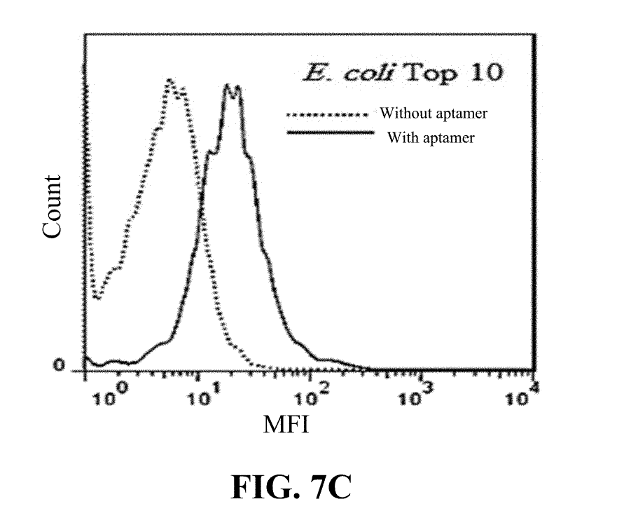

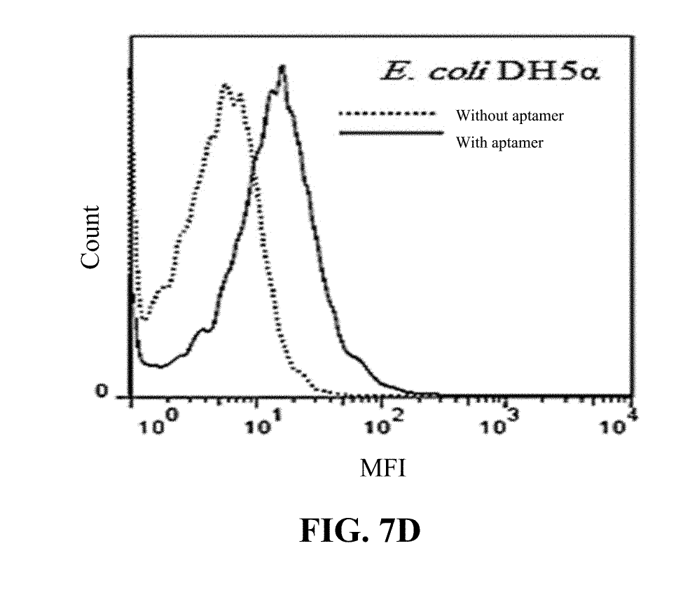

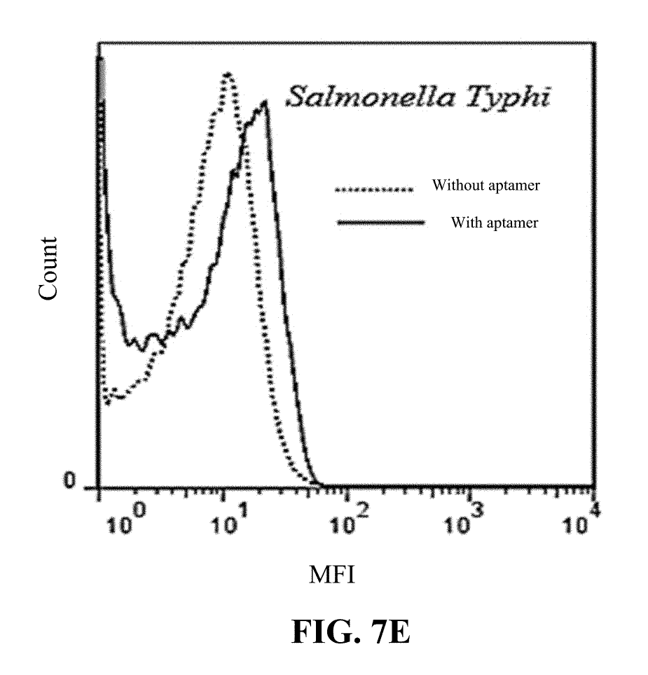

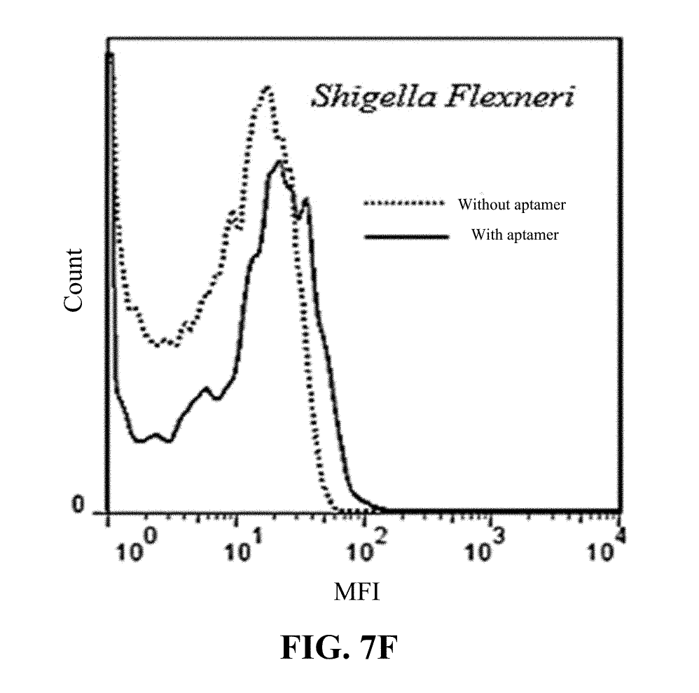

FIG. 7A shows binding affinity of the E. coli O157:H7 aptamer with a nucleotide sequence as set forth in SEQ ID No. 6 (AM-6) to E. coli 042 cells, consistent with one or more exemplary embodiments of the present disclosure. Referring to FIG. 7A, a low degree of cross-reactivity of the E. coli O157:H7 aptamer was observed for 042 E. coli cells with 4% of fluorescence intensity in comparison with the E. coli O157:H7 cells.

FIG. 7B shows binding affinity of the E. coli O157:H7 aptamer with a nucleotide sequence as set forth in SEQ ID No. 6 (AM-6) to E. coli K12 cells, consistent with one or more exemplary embodiments of the present disclosure. Referring to FIG. 7B, a low degree of cross-reactivity of the E. coli O157:H7 aptamer was observed for K12 E. coli cells with 2% of fluorescence intensity in comparison to the E. coli O157:H7 cells.

FIG. 7C shows binding affinity of the E. coli O157:H7 aptamer with a nucleotide sequence as set forth in SEQ ID No. 6 (AM-6) to E. coli TOP10 cells, consistent with one or more exemplary embodiments of the present disclosure. Referring to FIG. 7C, a moderate cross-reactivity of the E. coli O157:H7 aptamer was detected for Top 10 E. coli cells with 8% of fluorescence intensity in comparison to the E. coli O157:H7 cells.

FIG. 7D shows binding affinity of the E. coli O157:H7 aptamer with a nucleotide sequence as set forth in SEQ ID No. 6 (AM-6) to E. coli DH5α cells, consistent with one or more exemplary embodiments of the present disclosure. Referring to FIG. 7D, a moderate cross-reactivity of the E. coli O157:H7 aptamer was detected for DH5α E. coli cells with 7% of fluorescence intensity in comparison to the E. coli O157:H7 cells.

FIG. 7E shows binding affinity of the E. coli O157:H7 aptamer with a nucleotide sequence as set forth in SEQ ID No. 6 (AM-6) to Salmonella typhi cells, consistent with one or more exemplary embodiments of the present disclosure. Referring to FIG. 7E, a low degree of cross-reactivity of the E. coli O157:H7 aptamer was observed for Salmonella typhi cells with 0.6% of fluorescence intensity in comparison to the E. coli O157:H7 cells.

FIG. 7F shows binding affinity of the E. coli O157:H7 aptamer with a nucleotide sequence as set forth in SEQ ID No. 6 (AM-6) to Shigella flexneri cells, consistent with one or more exemplary embodiments of the present disclosure. Referring to FIG. 7F, a low degree of cross-reactivity of the E. coli O157:H7 aptamer was observed for Shigella flexneri cells with 1% of fluorescence intensity in comparison to the E. coli O157:H7 cells.

Referring again to FIGS. 7A-7F, the fluorescence intensities of other strains are significantly different from the 66.26% of fluorescence intensity for the E. coli O157:H7 cells. Therefore, the cross-reactivity results confirm the high specificity of the E. coli O157:H7 aptamer in capturing of the target strain of E. coli O157:H7 cells.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed. Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A method for detecting Escherichia coli (E. coli) O157:H7 in a sample comprising:
synthesizing an E. coli O157:H7 aptamer, the E. coli O157:H7 aptamer comprising a nucleic acid sequence as set forth in one of SEQ ID No. 1, SEQ ID No. 2, SEQ ID No. 3, SEQ ID No. 4, SEQ ID No. 5, and SEQ ID No. 6;
adding the E. coli O157:H7 aptamer to the sample allowing for formation of a complex by contact of the E. coli O157:H7 aptamer with any E. coli O157:H7 in the sample; and
determining the formation of the complex by detecting presence of the complex in the sample.

2. The method according to claim 1, wherein the E. coli O157:H7 aptamer comprises the nucleic acid sequence as set forth in SEQ ID No. 6.

3. The method according to claim 1, wherein synthesizing the E. coli O157:H7 aptamer comprises:
identifying the E. coli O157:H7 aptamer, the E. coli O157:H7 aptamer comprising the nucleic acid sequence as set forth in one of SEQ ID No. 1, SEQ ID No. 2, SEQ ID No. 3, SEQ ID No. 4, SEQ ID No. 5, and SEQ ID No. 6; and
amplifying the E. coli O157:H7 aptamer.

4. The method according to claim 3, wherein identifying the E. coli O157:H7 aptamer comprises identifying the aptamer using the whole-cell systematic evolution of ligands by exponential enrichment (cell-SELEX) method.

5. The method according to claim 3, wherein amplifying the E. coli O157:H7 aptamer comprises amplifying the aptamer using polymerase chain reaction (PCR).

6. The method according to claim 1, wherein the E. coli O157:H7 aptamer has between 118 and 123 nucleotides.

7. The method according to claim 1, wherein adding the E. coli O157:H7 aptamer to the sample allowing for formation of the complex by contact of the E. coli O157:H7 aptamer with any E. coli O157:H7 in the sample comprises adding an E. coli O157:H7 aptamer solution with a concentration more than 300 pM to the sample.

8. The method according to claim 1, wherein determining the formation of the complex by detecting presence of the complex in the sample comprises detecting the presence of the complex using one of polymerase chain reaction (PCR), Real-Time PCR, an affinity tag, fluorescence, chemiluminescence, nuclear magnetic resonance, enzyme-linked immunosorbent assay (ELISA), and combinations thereof.

9. The method according to claim 1, wherein determining the formation of the complex by detecting presence of the complex in the sample comprises detecting the quantity of the complex in the sample using an aptasensor.

10. The method according to claim 1, wherein the sample comprises one of a physiological sample and a cultural sample.

11. The method according to claim 10, wherein the physiological sample comprises one of saliva, urine, and blood.

12. The method according to claim 11, wherein the physiological sample comprises one of a human physiological sample or an animal physiological sample.

13. The method according to claim 10, wherein the cultural sample comprises one of a water sample or a food sample.

14. An E. coli O157:H7 aptamer for binding to Escherichia coli (E. coli) O157:H7, the E. coli O157:H7 aptamer comprising a nucleic acid sequence as set forth in one of SEQ ID No. 1, SEQ ID No. 2, SEQ ID No. 3, SEQ ID No. 4, SEQ ID No. 5, and SEQ ID No. 6.

15. The E. coli O157:H7 aptamer according to claim 14, wherein the E. coli O157:H7 aptamer has a dissociation constant (K_{d}) between 39.8 pM and 175.4 pM.

16. The E. coli O157:H7 aptamer according to claim 14, wherein the E. coli O157:H7 aptamer comprises between 118 and 123 nucleotides.

17. The E. coli O157:H7 aptamer according to claim 14, wherein the E. coli O157:H7 aptamer binds to one of a cell wall and a cell membrane of the E. coli O157:H7.

18. The E. coli O157:H7 aptamer according to claim 14, wherein the E. coli O157:H7 aptamer is linked to one of a fluorescent tag, an affinity tag, a magnetic tag, a therapeutic agent, and combinations thereof.

19. A method for detecting Escherichia coli (E. coli) O157:H7 in a sample comprising:
adding an E. coli O157:H7 aptamer to the sample allowing for formation of a complex by contact of the E. coli O157:H7 aptamer with any E. coli O157:H7 in the sample, the E. coli O157:H7 aptamer comprising a nucleic acid sequence as set forth in one of SEQ ID No. 1, SEQ ID No. 2, SEQ ID No. 3, SEQ ID No. 4, SEQ ID No. 5, and SEQ ID No. 6; and
determining the formation of the complex by detecting the presence of the complex in the sample.

20. The method according to claim 19, wherein the E. coli O157:H7 aptamer comprises the nucleic acid sequence as set forth in SEQ ID No. 6.
